(19) United States
(12) Patent Application Publication
(10) Pub. No.: US 2012/0316390 A1
(43) Pub. Date: Dec. 13, 2012

(54) STIMULATOR WITH FLEXIBLE ELEMENTS (VARIANT EMBODIMENTS)

(76) Inventor: Sergei E. Topolev, Sestroretsk (RU)

(21) Appl. No.: 13/512,481

(22) PCT Filed: Nov. 25, 2010
(86) PCT No.: PCT/RU2010/000726

§ 371 Date: May 29, 2012

(30) Foreign Application Priority Data
Nov. 26, 2009 (RU) 2009145050

(51) Int. Cl. A61F 5/00; A61H 1/00

(52) U.S. Cl. 600/38; 601/46

(57) ABSTRACT

The invention relates to devices for private use which enhance arousal and facilitate orgasm, and can be used for massaging the genital organs and correcting sexual inadequacy. The object of the invention is to enhance sexual sensations, maintain and sustain arousal, facilitate orgasm, and improve blood circulation in the genital organs. The essence of the invention is a device with flexible and elastic elements, which increase a zone of stimulation due to their elasticity and allow the device to be shaped and sized to be suitable for frictions in the vagina due to its flexibility. The claimed invention makes it possible to achieve intense and continuous stimulation of external or internal genital organs when performing frictional vaginal stimulation.

### BACKGROUND OF THE INVENTION

The invention relates to medical equipment, sexuality section, in particular, to devices for personal use, intensifying arousal and facilitate the achievement of orgasm, and can be used to massage the sexual organs and correction of sexual disharmony.

Being present invention is a device with a flexible and elastic elements, which are due to increased elasticity of a zone of stimulation, but at the expense of flexibility allows the device have the shape and size suitable to move into a vagina. The claimed invention solves a problem of a strong and continuous stimulation of external or internal friction in a genital vaginal stimulation.

The analogue of the first embodiment is a device for sexual stimulation, which has one base and two elongated stimulating element for simultaneous vaginal and anal stimulation. The disadvantage of this device is the inability or soreness at the same time introducing elements of both stimulating the vagina, as well as their shape and physical properties do not allow to significantly increase the stimulating effect of the vagina.

The analogue of the second embodiment is a device for sexual stimulation phallic shape, made of elastic material and a hollow sealed with an external compressor, providing an increase in the size of the device. The disadvantage of this device is not dependent on the phase displacement of the vagina form of the device, only the external variable supercharger.

The analogue of the third embodiment is a dildo with a clitoral stimulator such as “rabbit” or ring for clitoral stimulator with a penis and additionally equipped with a vibrator. The disadvantage of such devices is the momentary contact with the clitoris clitoral element for moving the elongated dildo or penis into the vagina.

The analogue of a fourth embodiment of a device for vaginal stimulation, performed as a dildo or a penis in the nozzles in the form of a tube (sleeve) or a condom, and having a protruding on the surface of elongated flexible elements of the stimulus, made in the form of “antennae”. The disadvantage of such devices is short and slight stimulation of erogenous zones.

### SUMMARY OF THE INVENTION

An essential feature of the first embodiment of the invention is a longitudinal split in the elongated shape of the device phallic, some or all of which, bent in the direction of the longitudinal axis and made flexible and elastic, for example, from rubber-like material, or contain an internal elastic core, or are able to flex relative to the longitudinal axis of the device, which contains, for example, internal hinges.

An additional important feature is the presence of a locking device which allows a bend or change the incentive to fix an item in a straightforward manner, for example, made in the form of a ring being moved along the device, or in a lock profile, or electromagnetic elements on the sides of mating, or as an internal telescopic rods.

The technical result is the first embodiment is the vaginal extension without the extension of the vaginal opening with frictional stimulation.

The task of the first embodiment is to strengthen the stimulating effect of the increase in the vaginal area stimulated by its expansion, without a considerable expansion of the vagina, increasing sexual arousal and enhanced stimulation of the inner vaginal erogenous zones, in particular the G-spot, prevention of sexual disorders.

An essential feature of the second embodiment is the presence of a longitudinal cross-cutting long slits in the phallic-shaped device made of flexible and elastic material that allows you to bend the separated parts in opposite directions relative to the longitudinal axis of the device under longitudinal compression device. An additional important feature is the presence of the device that allows you to capture the division of each other.

The technical result of the second embodiment is the vaginal extension without the extension of a vaginal opening with frictional stimulation.

The task of the second embodiment is to enhance the stimulating effect of increasing the useful surface of the device by stimulating its expansion, without a considerable expansion of the vagina, increasing sexual arousal and enhanced stimulation of the inner vaginal erogenous zones, in particular the G-spot, prevention of sexual disorders.

An essential feature of the third embodiment is one or more flexible and resilient clamping elements on the dildo or ring (nozzle) to the penis, clitoral stimulator connected to, and provide a constant clitoral stimulator to the pressing of the outer genitals with vaginal stimulation dildo or penis, put on a device for which the clitoral stimulator has a hole or slot into which is placed a dildo or penis. Additional important features are: the presence of the devices or form of execution clitoral stimulator, providing improved moving clitoral stimulator on the dildo or penis, clitoral stimulator unit fixed on the dildo or ring (nozzle) to the penis, the device providing the angular rotation of the clitoral element when moving along a dildo, and forms of execution, which provides radial compression ring or attachment for the penis when pressed to clitoral pacemaker.

The technical result is the third embodiment is to continuously pressed against clitoral stimulator to the external genitals with vaginal stimulation friction.

The task of the third embodiment is to enhance the stimulating effect of continuous exposure clitoral stimulator with external genitals with stimulation of a vagina back and forth movements, enhancing sexual excitement, the prevention of sexual disorders.

The essential feature of the fourth embodiment is one or more tape of flexible and elastic elements of the stimulus, fixed longitudinally on the device phallic shape, nozzle penis or condom, one edge, so that they can bend to the device when moving through the narrowing of the vaginal opening, and restoring its shape, stimulate outer labia and clitoris when you move the device into the vagina.

The technical result is the fourth embodiment is to better stimulate the female external genitalia, including the labia minora and the clitoris, the continuous contact with them during frictional stimulation of the vagina.

The task of the fourth embodiment is to enhance the stimulation of the erogenous zones of the external female genital organs, facilitating the achievement of orgasm, the more vivid sexual sensations, the prevention of sexual disorders.

### BRIEF DESCRIPTION OF THE DRAWINGS

FIG. 1 shows a longitudinal section of the first embodiment of the invention in the form of a dildo to the body, with a forked head-on in the open position and the locking ring 2, installed in the rear.

In FIG. 2 shows a longitudinal section of the first embodiment of the invention in the form of a dildo to the body, with a forked head-on in the closed state and the locking ring 2, installed in the front.

FIG. 3 shows a longitudinal section of the first embodiment of the invention in the form of a dildo to the body, with a forked head-on in the open position and a locking profile 3 on the inner sides of the forked part.

FIG. 4 shows a longitudinal section of the first embodiment of the invention in the form of a dildo to the body with a forked front of the open-loop and internal cavities, which are installed inside the sliding rod 4.

FIG. 5 shows a longitudinal section of the first embodiment of the invention in the form of a dildo to the body with a forked head-on in the open position, an external focus and internal cavities 5, which are installed inside a rigid hinge elements 6, which is set to move the pushing member 7.

FIG. 6 shows a longitudinal section of the second embodiment of the invention in the form of a hollow body with a dildo 8, 9 and supercharger working part 10.

FIG. 7 shows a longitudinal section of a third embodiment of the invention in the form of a dildo to the body 11 and the stimulating element 12.

FIG. 8 shows a cross section of a third embodiment of the invention in the form of a dildo to the body 11 and stimulating elements 12.

### DETAILED DESCRIPTION OF THE PREFERRED EMBODIMENTS

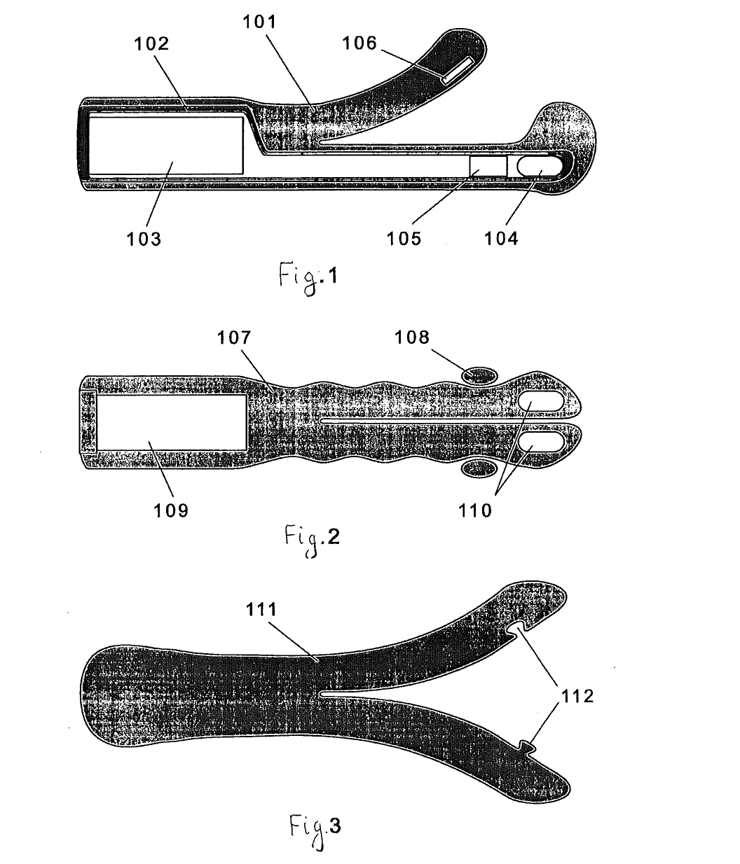

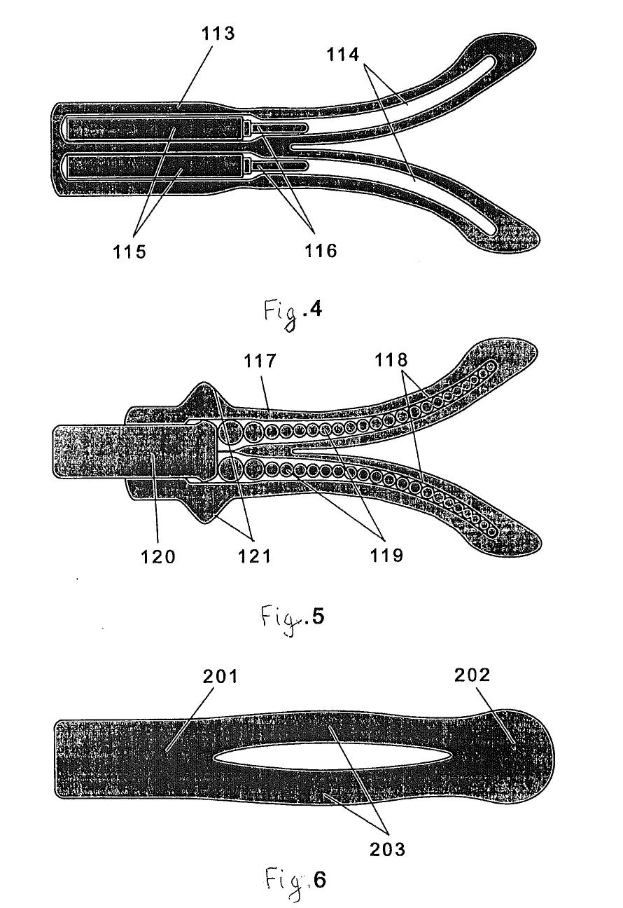

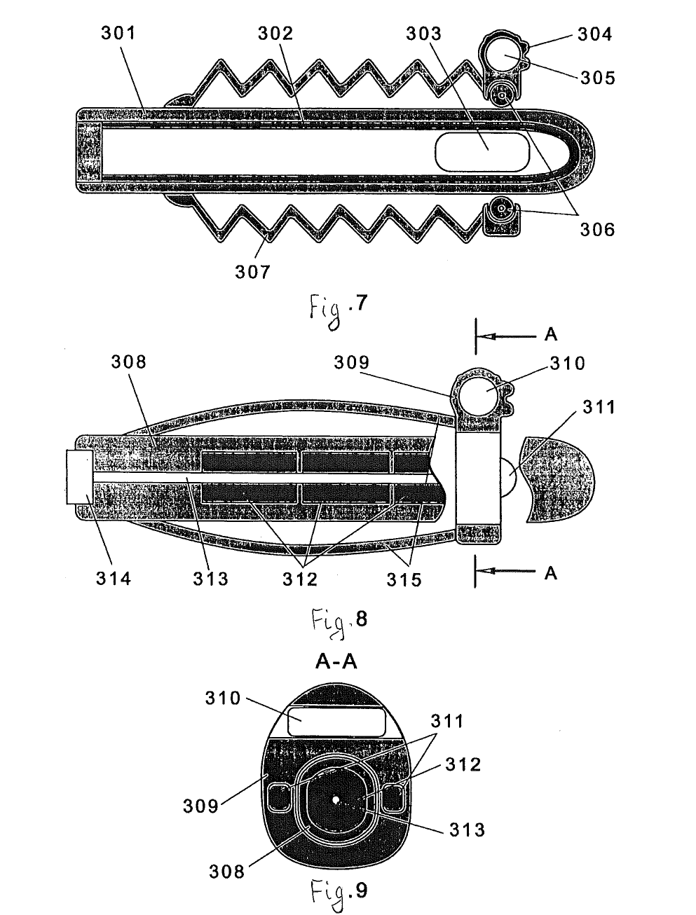

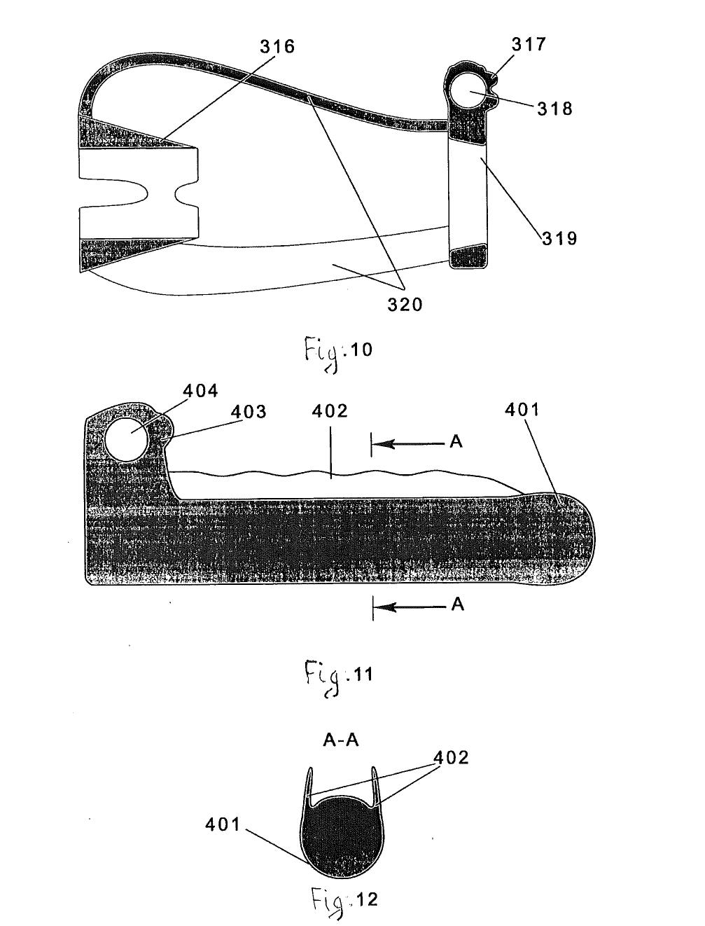

Implementation of the first embodiment shown in FIG. 1, FIG. 2, made in the form of a bifurcated dildo with a locking ring 2 and the wave-like profile, designed to adjust the speed of bending stimulus elements. In FIG. 3 shows the performance of the device with a locking profile on the three interlocking (conjugate) elements of the stimulus side. In FIG. 4 shows the performance of devices with internal pull-rod 4, in which you move, change bend stimulus elements. With the introduction of the device into the vagina loose ends stimulus elements are bent towards each other, without impeding progress inside the device, and with further input into the vagina loose ends are bent in opposite directions from each other. In FIG. 5 shows the performance of the device, with an emphasis 5 and 6 internal hinges that are moving along the change curve of the cavities enabling elements for which the device has a plunger 7, which is when you move deeper into the device joints, causing curving stimulating element, while the device pressed emphasis to the external genitalia. The device is made of rubber-like material such as silicone rubber, soft plastic, and may contain parts made of metal plastic or other similar materials. In addition, the device may comprise one or more vibrators, clitoral, anal stimulator, or a combination of them, have a catalytic surface relief, executed in the anatomical form as attachment to the penis, as well as a mount for fixing to the body.

Implementation of the second embodiment shown in FIG. 6, made in the form of a hollow cone with a dildo 8 supercharged 9 in rear end walls and the subtle working of the 10 front end. Dildo filled with air, liquid or gel and is made tight, and with the possibility of filling and sealing. With the introduction of the device in the vagina, the supercharger is compressed by pressing to the external genitals, with the front-end devices is expanding. The compressor can be performed in a cone shape with segments of varying thickness for the radial compression of the thickened part of the supercharger, or in the form of a corrugated tube. The compressor can be manual, mechanical, electromechanical or hydraulic drive.

The device is made of rubber-like material such as silicone rubber, soft plastic, and may contain parts made of metal, plastic and other similar materials. In addition, the device may comprise one or more vibrators, clitoral, anal stimulator, or a combination of them, have a catalytic surface relief, executed in the anatomical form as attachment to the penis, as well as a mount for fixing to the body. Implementation of the third embodiment shown in FIG. 7, FIG. 8, in the shape of a dildo 11, with two tape stimulating elements 12 mounted along the axis tangentially dildo. When moving into the vagina, stimulating elements disclosed stimulate the labia and clitoris, and the passage of the vaginal opening bend, clinging to the body unit. Incentive items can have a contoured surface, and a variable height, or thickness. The device is made of rubber-like material such as silicone rubber, soft plastic, and may contain parts made of metal plastic or other similar materials. In addition, the device may comprise one or more vibrators, clitoral, anal stimulator, or a combination of them, have a catalytic surface relief, executed in the anatomical form as attachment to the penis, as well as a mount for fixing to the body.

1. A device for sexual stimulation, comprising a base, having a shape and size suitable for holding a hand, or to enter into a vagina, or to connect parts of a body directly or through auxiliary elements, elongated vaginal member having a shape and size suitable for insertion into a vagina, and at least one elongated flexible and resilient stimulating member made, for example, silicone rubber, which may further comprise an elastic core, or a hinge structure, one end of which is fixed on the basis of vaginal member in a direction close to a longitudinal axis or plane of the vaginal member, wherein a free end of the stimulating member is bent in a direction close to perpendicular to a longitudinal axis or plane of the vaginal member, and when it is flexed to vaginal member that can be made at least one similar stimulating member with the opposite curvature, they form a common phallic shape and size suitable for insertion into a vagina, and a force of elasticity and flexural stimulating member provide an expansion of a vagina, and allow him to bend to the vaginal member or to another stimulating member as you move through a narrowing of a vaginal opening, additionally, the device can be accomplished with a possibility of fixing the free end of the stimulating member to another stimulating member or vaginal member, and/or with the ability to control the bending and/or elastic force of the stimulating member.

2. The device of accordance with claim 1, wherein the executed wholly or partly of rubber-like material such as silicone rubber.

3. The device of accordance with claim 1, wherein further comprises at least one vibrating device.

4. The device of accordance with claim 1, wherein further comprises at least one external stimulating element, such as a clitoral stimulator, which may additionally contain at least one vibrating device.

5. The device of accordance with claim 1, wherein the control device further comprises a bend, and/or elastic force, and/or fixing the free end of the stimulating member, what it contains, for example, on the side facing the vaginal member made a similar stimulating member, at least one locking device, made, for example, in the form of incentive to lock the profile element, and the response profile to another stimulating member, in conjunction with an effort which is fixing them, or contains at least one electromagnetic device, for example, in the vaginal cell, and magnetic metal element in stimulating element that is attracted to the electromagnetic device when it is activated, and captures both elements, or the device contains an additional element in the shape of a ring or a cuff with the ability to navigate on the stimulating member and the vaginal part member and or other stimulating member, in which you move along the longitudinal axis of the free end of the stimulating member, there is a connection of adjacent elements to each other, or the device is a hinge mechanism, which stimulates the part of the base element made of rubber-like material and has an internal cavity in which there are mobile, mostly spherical, rigid structure and a pusher attached to the base, as well as, the protrusion on the rubber part of the base, which provides stimulating member in bending in different directions, and subjected to a follower, or telescopic device includes a core set mostly in the ground when moving a sliding part whose internal cavity stimulating member is its bending.

6. The device of accordance with claim 1, wherein the base and vaginal member contain a common element of the rigid body, which may additionally contain at least one electro-mechanical vibratory device having a size in length from 50 to 300 mm, preferably 190 mm, with a predominantly, a circular cross section for the base and, mostly, a semi-circular cross-section for vaginal element with a diameter of 5 to 80 mm, preferably 30 mm, and at least one stimulating member in length from 30 to 200 mm, preferably 70 mm, preferably semi-circular cross-section diameter from 15 to 100 mm, preferably 40 mm, with an outer radial curvature, so that the distance from the inner side of the free end to the vaginal member ranges from 30 to 100 mm, preferably 50 mm, and the stimulating member is a wrapper element with a thickness of the vaginal member 1 to 20 mm, preferably 5 mm, of a rubber material such as silicone hardness of 10 to 80, preferably 45 Shore units, and additionally with a wrapper for the foundation, or part thereof, in addition, stimulating member may further comprise a vibrator, and the device further fixation device may include stimulating member.

7. The device of accordance with claim 1, wherein is made of rubber-like material such as silicone rubber hardness of 10 to 80, preferably 45 Shore units, and contains at least two approximately equal in size and shape of stimulating member length from 30 to 150 mm, preferably 90 mm, preferably semi-circular cross-section diameter of 15 to 100 mm, preferably 40 mm, with a bend in different directions, one of which is the vaginal part of the outer radial curvature, so that the distance between the internal sides of the free ends of the stimulating members from 30 to 200 mm, preferably 100 mm, and additionally at least one incentive element can contain a vibrator, preferably shifted to the free end, and the device may further comprise a device fixation stimulus components with each other.

8-11. (canceled)

12. A device for sexual stimulation, comprising the base, having shape and size suitable for holding a hand, or to enter into a vagina, or to connect parts of a body directly, or through the support elements, vaginal member with the central axis, which can be executed and have an elongated shape and size suitable for insertion into a vagina or in the form of a nozzle or a ring-shaped and size suitable for placing and fixing on a penis, and at least a clitoral stimulating member having a shape and size suitable for stimulation of a clitoris and/or external genitalia, wherein in that the clitoral element is arranged to move along the central axis of the vaginal member, which contains, for example, a hole or a slit having the shape and size suitable for placement inside the vagina or penis element with a nozzle or a ring, or has the form and/or the support element, allowing the element to fix the clitoral area of the clitoris and external genitalia when you move an item or vaginal penis with a nozzle or a ring into the vagina, and, clitoral and spring-loaded element is connected to the base, which contains the pressure element, made, for example, in the form of a compression spring or a corrugated elastic tube, or at least one elongated flexible and elastic element made, for example, in the form of ribbons or strips, and may additionally contain at least one device capable of moving vaginal member or a penis with a nozzle or a ring, made, for example, in the form of a roller or rollers on the side facing the central axis of the vaginal member, as well as the device may contain a locking device that allows you to capture an element on the basis of the clitoral or vaginal entry.

13. The device of accordance with claim 12, wherein the executed wholly or partly of rubber-like material such as silicone rubber.

14. The device of accordance with claim 12, wherein it additionally contains at least one vibrating device.

15. The device of accordance with claim 12, wherein is made of rubber-like material, has an elongated vaginal member having a shape and size suitable for insertion into the vagina, with installed inside the hard shell, which may additionally contain a vibrating device, and clitoral element with a hole, providing free movement along the vaginal element, and may additionally contain a side facing the element vaginal clips, for which the clitoral element is a hard shell, or grooves that ensure the distribution of lubricant, and a flexible and resilient clamping element in the shape of the corrugated elastic tube, and also, the device may further comprise retainer, in the shape of the profile of the castle on the clitoral and the mating element on the bottom, with the strong connection which provides clitoral fixation element on the bottom.

16. The device of accordance with claim 12, wherein the following element to the elongated vaginal, preferably cylindrical in shape, containing at least one longitudinal guide, performed, for example, in the shape of the grooves, projections or plane, which can be made curved, for example, undulating, and clitoral element with an aperture having a shape which provides movement along the vaginal fixation of the radial element of the angular rotation relative to the guide for what on the side facing the central axis of the vaginal element contains, for example, at least one protrusion, the groove or plane, and, clitoral element is connected to the base, with the possibility of radial angular rotation axis relative to the base element of the vaginal, at least two flexible and resilient clamping elements made in the form of strips and clitoral element may further comprise on the side facing away from the base at least two rollers or two elongated flexible and elastic element in a direction close to the central axis of the vaginal element to ensure the rotation of the clitoral element of external female genital organs and vaginal additional element may be arranged to change the curvilinear guide, which can be made of a flexible and resilient material such as silicone rubber, and contain an internal core with attached to it along the longitudinal axis of the rigid elements connected to the vaginal part of which, when rotated in different directions along the central axis of the shift of the longitudinal vaginal element by changing the linear guide.

17. The device of accordance with claim 12, wherein it contains ring-shaped base element in the form of a vaginal ring that has the shape and dimensions suitable for placing and fixing on the penis, the clitoral element with a hole that can be cut-through, and additionally may contain on the inside at least one roller, which provides the size of the free movement along the penis, and may additionally contain a contoured surface and/or vibration device, and at least two, preferably three, flexible and elastic clamping element made in the form of strips, fixed uniformly with respect to the central axis of the vaginal element, and, on the basis of a direction close to perpendicular to the axis of the central element of the vaginal and clitoral element in a direction close to parallel to the central axis of the vaginal element, for which these elements are bending, as well as the device additionally may contain a latch, lock in the shape of the profile element in the clitoral and the mate on the ground or vaginal part, with the strong connection which is fixing them.

18. The device of accordance with claim 12, wherein the vaginal member is in the form of tapered rings at least two segments connected by a flexible jumper and the narrow side facing to the clitoral element that has a hole, for example, tapered, providing a radial contraction of the segments ring when placed in a vaginal item.

19. A device for sexual stimulation, comprising a base, having a shape and size suitable for holding a hand, or to enter into a vagina, or to connect parts of a body directly or through auxiliary elements, elongated vaginal member having a shape and size suitable for insertion into a vagina, and at least one flexible, resilient stimulating member made, for example, silicone rubber, wherein it is made in the form of ribbons or strips, and may additionally contain a contoured surface, mounted on one edge of a longitudinal vaginal member so that when its bending element to form a vaginal general form suitable for insertion into a vagina, for example, in a direction close to the tangential direction of the longitudinal section of the vaginal member, with the flexibility and elasticity of the stimulating member allow it to stimulate a external genitals and bend to the vaginal element when moving a narrowing of a vaginal opening, and, optionally, stimulating member may contain a fixing device for vaginal entry.

20. The device of accordance with claim 19, wherein the executed wholly or partly of rubber-like material such as silicone rubber.

21. The device of accordance with claim 19, wherein it additionally contains at least one vibrating device.

22. The device of accordance with claim 19, wherein it additionally contains at least one external stimulus element, such as the clitoral stimulator, which additionally may contain at least one vibrating device.

23. The device of accordance with claim 19, wherein the vaginal member is designed as a cap on the penis or the condom.

24. The device of accordance with claim 19, wherein is adapted to lock stimulating member to vaginal member, which stimulates the element contains the longitudinal profile of the castle on the side facing the vaginal member closer to the free edge, and the return profile on vaginal cell, ensuring their fixation when pressed against the strong.

25. (canceled)
